(19) United States
(12) Patent Application Publication
(10) Pub. No.: US 2013/0210846 A1
(43) Pub. Date: Aug. 15, 2013

(54) USE OF NOVEL PAN-CDK INHIBITORS FOR TREATING TUMORS

(71) Applicants: Ulrich Lücking, Berlin (DE); Gerhard Siemeister, Berlin (DE); Antje Margret Wengner, Berlin (DE)

(72) Inventors: Ulrich Lücking, Berlin (DE); Gerhard Siemeister, Berlin (DE); Antje Margret Wengner, Berlin (DE)

(73) Assignee: BAYER INTELLECTUAL PROPERTY GMBH, MONHEIM (DE)

(21) Appl. No.: 13/638,833

(22) PCT Filed: Mar. 28, 2011
(86) PCT No.: PCT/EP2011/054733

§ 371 Date: Mar. 13, 2013

(30) Foreign Application Priority Data
Apr. 1, 2010 (DE) 102010014426.6

(51) Int. Cl. C07D 239/47; C07D 239/48

(52) U.S. Cl. CPC C07D 239/47; C07D 239/48
USPC 514/272; 514/275

(57) ABSTRACT

The invention relates to the use of selected sulfoximine-substituted anilinopyrimidine derivatives of the formula (I) for treating tumors.

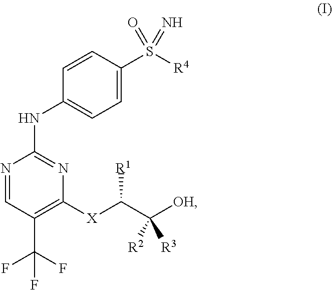

The present invention relates to the use of novel pan-CDK inhibitors for treating tumors.

The novel pan-CDK inhibitors are selected sulfoximine-substituted anilinopyrimidine derivatives.

The novel pan-CDK inhibitors and processes for their preparation are described in the PCT application PCT/EP2009/007247, the disclosure of which is referred to in the present application and which is incorporated into this application by reference.

Cyclin-dependent kinases (CDKs) are a family of enzymes playing an important role in the regulation of the cell cycle, and they are therefore a particularly interesting target for the development of small inhibitory molecules. Selective inhibitors of the CDKs can be used for treating cancer or other disorders caused by a disturbed cell proliferation.

Pyrimidines and analogs have already been described as active compounds, for example 2-anilino-pyrimidines as fungicides (DE 4029650) or substituted pyrimidine derivatives for the treatment of neurological or neurodegenerative disorders (WO 99/19305). Highly diverse pyrimidine derivatives, for example 2-amino-4-substituted pyrimidines (WO 01/14375), purines (WO 99/02162), 5-cyano-pyrimidines (WO 02/04429), anilinopyrimidines (WO 00/12486) and 2-hydroxy-3-N,N-dimethylaminopropoxypyrimidines (WO 00/39101) have been described as CDK inhibitors.

WO 02/096888 and WO 03/076437 in particular disclose pyrimidine derivatives having inhibitory action with respect to CDKs.

Compounds that contain a phenylsulfonamide group are known as inhibitors of human carboanhydrases (in particular carboanhydrase-2) and are used as diuretics, inter alia for treating glaucoma. The nitrogen atom and the oxygen atoms of the sulfonamide bind via hydrogen bonds to the zinc²⁺ ion and the amino acid Thr 199 in the active center of the carboanhydrase-2 and thus block their enzymatic function (A. Casini, F. Abbate, A. Scozzafava, C. T. Supuran, Bioorganic. Med. Chem. Lett. 2003, 1, 2759). The clinical use of CDK inhibitors containing a phenylsulfonamide group could be restricted owing to a possible inhibition of carboanhydrases and a resulting side-effect spectrum.

Examples of active sulfoximine compounds are sulfonimidoyl-modified triazoles as fungicides (H. Kawanishi, H. Morimoto, T. Nakano, T. Watanabe, K. Oda, K. Tsujihara, Heterocycles 1998, 49, 181) or arylalkylsulfoximines as herbicides and pesticides (Shell International Research, Ger. P. 2 129 678).

WO 2005/037800 discloses open sulfoximine-substituted anilinopyrimidine derivatives as inhibitors of cyclin-dependent kinases. Examples given are structures which, in the 5-position of the pyrimidine, are either unsubstituted or substituted by halogen, in particular by bromine None of the structures specifically disclosed had a 5-trifluoromethyl substituent.

Based on this prior art, it is an object of the present invention to provide compounds which not only potently inhibit CDK but also effectively inhibit tumor growth. Potent CDK inhibition is a necessary, but insufficient precondition for effective tumor inhibition. The latter requires further properties of the structures, for example the capability to penetrate into the tumor cell.

It has now been found that compounds of the general formula (I)

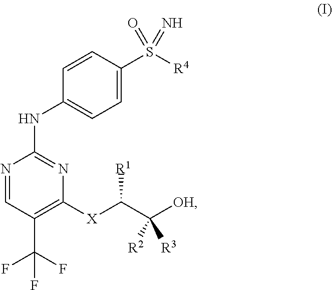

in which
X represents —O— or —NH—, and
R¹ represents a methyl, ethyl, propyl or isopropyl group, and
R² and R³ independently of one another represent hydrogen, a methyl or ethyl group, and
R⁴ represents a C₁-C₆-alkyl group or a C₃-C₇-cycloalkyl ring,
and their physiologically acceptable salts, diastereomers and enantiomers
not only inhibit CDK in a potent manner but also inhibit tumor growth particularly effectively.

Compounds in which X represents —O— are summarized by formula (Ia).

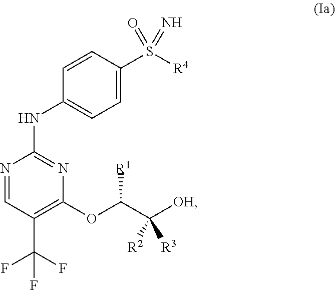

Compounds in which X represents —NH— are summarized by formula (Ib).

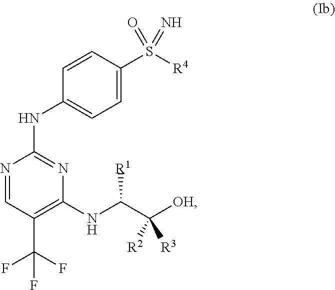

The application is based on the following definitions:

### C₁-C₆ Alkyl

A C₁-C₆-alkyl group is defined in each case as a straight-chain or branched alkyl radical such as, for example, a methyl, ethyl, propyl, isopropyl, butyl, isobutyl, sec-butyl, tert-butyl, pentyl, isopentyl or a hexyl radical.

### C₃-C₇-Cycloalkyl

A C₃-C₇-cycloalkyl ring is defined as a cyclopropyl, cyclobutyl, cyclopentyl, cyclohexyl or a cycloheptyl ring.

In the general formula (I), X may represent —O— or —NH—.

Preferably, X represents —O—.

In the general formula (I), R¹ may represent a methyl, ethyl, propyl or isopropyl group.

Preferably, R¹ represents a methyl group.

In the general formula (I), R² and R³ independently of one another may represent hydrogen, a methyl or an ethyl group.

Preferably, R² and R³ independently of one another represent hydrogen or a methyl group.

Particularly preferably, R² represents a methyl group and R³ represents hydrogen or a methyl group.

In the general formula (I), R⁴ represents a C₁-C₆-alkyl radical or a C₃-C₇-cycloalkyl ring.

Preferably, R⁴ represents a methyl or an ethyl group or represents a cyclopropyl ring.

A preferred sub-group of the compounds according to the general formula (I) are compounds in which

X represents —O— or —NH—, and
R¹ represents a methyl group, and
R² represents a methyl group, and
R³ represents hydrogen or a methyl group, and
R⁴ represents a methyl or an ethyl group or represents a cyclopropyl ring,
and their physiologically acceptable salts, diastereomers and enantiomers.

Most preference is given to the use according to the invention of the following individual compounds, and to their enantiomers, diastereomers and physiologically acceptable salts:
(RS)—S-cyclopropyl-S-(4-{[4-{[(1R,2R)-2-hydroxy-1-methylpropyl]oxy}-5-(trifluoromethyl)-pyrimidin-2-yl]amino}phenyl)sulfoximide,
(RS)—S-(4-{[4-{[(1R,2R)-2-hydroxy-1-methylpropyl]oxy}-5-(trifluoromethyl)pyrimidin-2-yl]-amino}phenyl)-S-methylsulfoximide,
(RS)—S-(4-{[4-{[(R)-2-hydroxy-1,2-dimethylpropyl]oxy}-5-(trifluoromethyl)pyrimidin-2-yl]amino}-phenyl)-S-methylsulfoximide,
(RS)—S-cyclopropyl-S-(4-{[4-{[(1R,2R)-2-hydroxy-1-methylpropyl]amino}-5-(trifluoromethyl)-pyrimidin-2-yl]amino}phenyl)sulfoximide,
(RS)—S-cyclopropyl-S-(4-{[4-{[(R)-2-hydroxy-1,2-dimethylpropyl]amino}-5-(trifluoromethyl)-pyrimidin-2-yl]amino}phenyl)sulfoximide,
(RS)—S-ethyl-S-(4-{[4-{[(1R,2R)-2-hydroxy-1-methylpropyl]amino}-5-(trifluoromethyl)pyrimidin-2-yl]amino}phenyl)sulfoximide,
(RS)—S-ethyl-S-(4-{[4-{[(R)-2-hydroxy-1,2-dimethylpropyl]amino}-5-(trifluoromethyl)pyrimidin-2-yl]amino}phenyl)sulfoximide,
(RS)—S-(4-{[4-{[(1R,2R)-2-hydroxy-1-methylpropyl]amino}-5-(trifluoromethyl)pyrimidin-2-yl]-amino}phenyl)-S-methylsulfoximide,
(RS)—S-(4-{[4-{[(1R)-2-hydroxy-1,2-dimethylpropyl]amino}-5-(trifluoromethyl)pyrimidin-2-yl]-amino}phenyl)-S-methylsulfoximide.

The present invention also embraces the use of the physiologically acceptable salts of the compounds.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropyl-amine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The present invention furthermore provides medicaments comprising at least one compound according to the invention and at least one or more further active compounds, in particular for the treatment and/or prophylaxis of tumour disorders.

The compounds according to the invention can act systemically and/or locally. For this purpose, they can be administered in a suitable manner, such as, for example, orally, parenterally, pulmonarily, nasally, sublingually, lingually, buccally, rectally, dermally, transdermally, conjunctivally, otically, as or as an implant or stent.

For these administration routes, the compounds according to the invention can be administered in suitable administration forms.

Suitable for oral administration are administration forms working according to the prior art, which release the compounds according to the invention rapidly and/or in modified form and comprise the compounds according to the invention in crystalline and/or amorphized and/or dissolved form, such as, for example, tablets (non-coated or coated tablets, for example coated with enteric, slowly dissolving or insoluble coats which control the release of the compound according to the invention), tablets which decompose rapidly in the oral cavity or films/wafers, films/lyophylizates, capsules (for example hard gelatin capsules or soft gelatin capsules), sugar-coated tablets, granules, pellets, powders, emulsions, suspensions, aerosols or solutions.

Parenteral administration can take place with circumvention of an absorption step (for example intravenous, intraarterial, intracardiac, intraspinal or intralumbar) or with involvement of an absorption (for example intramuscular, subcutaneous, intracutaneous, percutaneous or intraperitoneal). For parenteral administration, suitable administration forms are, inter alia, injection and infusion preparations in the form of solutions, suspensions, emulsions, lyophilizates or sterile powders.

Suitable for the other administration routes are, for example, pharmaceutical forms for inhalation (inter alia powder inhalers, nebulizers), nasal drops, nasal solutions, nasal sprays; tablets, films/wafers or capsules to be applied lingually, sublingually or buccally, suppositories, ear or eye preparations, vaginal capsules, aqueous suspensions (lotions, shake lotions), lipophilic suspensions, ointments, creams, transdermal therapeutic systems (such as, for example, patches), milk, pastes, foams, dusting powders, implants or stents.

The compounds according to the invention can be converted into the administration forms mentioned. This may take place in a manner known per se by mixing with inert non-toxic, pharmaceutically acceptable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and taste and/or odor corrigents.

The present invention furthermore provides medicaments comprising at least one compound according to the invention, usually together with one or more inert non-toxic, pharmaceutically suitable auxiliaries, and their use for the purposes mentioned above.

Formulation of the compounds according to the invention to give pharmaceutical products takes place in a manner known per se by converting the active compound(s) with the excipients customary in pharmaceutical technology into the desired administration form.

Auxiliaries which can be employed in this connection are, for example, carrier substances, fillers, disintegrants, binders, humectants, lubricants, absorbents and adsorbents, diluents, solvents, cosolvents, emulsifiers, solubilizers, masking flavors, colorants, preservatives, stabilizers, wetting agents, salts to alter the osmotic pressure or buffers.

Reference should be made in this connection to Remington's Pharmaceutical Science, 15th ed. Mack Publishing Company, East Pennsylvania (1980).

The pharmaceutical formulations may be

in solid form, for example as tablets, coated tablets, pills, suppositories, capsules, transdermal systems or
in semisolid form, for example as ointments, creams, gels, suppositories, emulsions or
in liquid form, for example as solutions, tinctures, suspensions or emulsions.

Auxiliaries in the context of the invention may be, for example, salts, saccharides (mono-, di-, tri-, oligo-, and/or polysaccharides), proteins, amino acids, peptides, fats, waxes, oils, hydrocarbons and derivatives thereof, where the auxiliaries may be of natural origin or may be obtained by synthesis or partial synthesis.

### Suitable for Oral or Peroral Administration are in Particular Tablets, Coated Tablets, Capsules, Pills, Powders, Granules, Pastilles, Suspensions, Emulsions or Solutions.

Suitable for parenteral administration are in particular suspensions, emulsions and especially solutions.

The present invention relates to the use of the compounds of the formulae (I) for the prophylaxis and therapy of tumor disorders.

The compounds of the formulae (I) can be used in particular for inhibiting or reducing cell proliferation and/or cell division and/or to induce apoptosis.

The compounds according to the invention are suitable in particular for the treatment of hyper-proliferative disorders such as, for example,
psoriasis,
keloids and other skin hyperplasias,
benign prostate hyperplasias (BPH),
solid tumors and
hematological tumors.

Solid tumors which can be treated in accordance with the invention are, for example, tumors of the breast, the respiratory tract, the brain, the reproductive organs, the gastrointestinal tract, the urogenital tract, the eye, the liver, the skin, the head and the neck, the thyroid gland, the parathyroid gland, the bones and the connective tissue and metastases of these tumors.

Hematological tumors which can be treated in accordance with the invention are, for example, multiple myelomas, lymphomas or leukemias.

Breast tumors which can be treated are, for example:
breast carcinomas with positive hormone receptor status
breast carcinomas with negative hormone receptor status
Her-2 positive breast carcinomas
hormone receptor and Her-2 negative breast carcinomas
BRCA-associated breast carcinomas
inflammatory breast carcinomas.

Tumors of the respiratory tract which can be treated are, for example,
non-small-cell bronchial carcinomas and
small-cell bronchial carcinomas.

Tumors of the brain which can be treated are, for example,
gliomas,
glioblastomas,
astrocytomas,
meningiomas and
medulloblastomas.

Tumors of the male reproductive organs which can be treated are, for example:
prostate carcinomas,
malignant testicular tumors and
penis carcinomas.

Tumors of the female reproductive organs which can be treated are, for example:
endometrial carcinomas
cervix carcinomas
ovarial carcinomas
vaginal carcinomas
vulvar carcinomas

Tumors of the gastrointestinal tract which can be treated are, for example:
colorectal carcinomas
anal carcinomas
stomach carcinomas
pancreas carcinomas
oesophagus carcinomas
gall bladder carcinomas
carcinomas of the small intestine
salivary gland carcinomas
neuroendocrine tumors
gastrointestinal stroma tumors

Tumors of the urogenital tract which can be treated are, for example:
urinary bladder carcinomas
kidney cell carcinomas
carcinomas of the renal pelvis and lower urinary tract

Tumors of the eye which can be treated are, for example:
retinoblastomas
intraocular melanomas

Tumors of the liver which can be treated are, for example:
hepatocellular carcinomas
cholangiocellular carcinomas

Tumors of the skin which can be treated are, for example:
malignant melanomas
basaliomas
spinaliomas
Kaposi sarcomas
Merkel cell carcinomas

Tumors of the head and neck which can be treated are, for example:
larynx carcinomas
carcinomas of the pharynx and the oral cavity

Sarcomas which can be treated are, for example:
soft tissue sarcomas
osteosarcomas

Lymphomas which can be treated are, for example:
non-Hodgkin lymphomas
Hodgkin lymphomas
cutaneous lymphomas
mantle cell lymphomas
lymphomas of the central nervous system
AIDS-associated lymphomas

Leukemias which can be treated are, for example:
acute myeloid leukemias
chronic myeloid leukemias
acute lymphatic leukemias
chronic lymphatic leukemias
hairy cell leukemias

Advantageously, the compounds of the formula (I) can be used for the treatment of breast carcinomas, in particular of hormone receptor negative, hormone receptor positive or BRCA-associated breast carcinomas, and also pancreas carcinomas, kidney cell carcinomas, malignant melanomas and other skin tumors, small-cell bronchial carcinomas, non-small-cell bronchial carcinomas, colorectal carcinomas, ovarial carcinomas, cervix carcinomas, prostate carcinomas, leukemias or lymphomas.

Particularly advantageously, the compounds of the formula (I) can be used for the treatment of breast carcinomas, in particular estrogen receptor-negative breast carcinomas, ovarial carcinomas, including

in particular cisplatin-resistant ovarial carcinomas,
colorectal carcinomas, small-cell bronchial carcinomas or
cervix carcinomas, including in particular multidrug-resistant cervix carcinomas.

These disorders are well-characterized in man, but also exist in other mammals.

The invention provides the use of the compounds of the general formula (I) according to the invention as medicaments for treating tumors.

The invention furthermore provides the use of the compounds of the general formula (I) according to the invention for preparing medicaments for treating tumors.

The invention furthermore provides the use of the compounds according to the invention for treating disorders associated with proliferative processes.

The compounds according to the invention can be employed by themselves or, if required, in combination with one or more other pharmacologically active substances, as long as this combination does not lead to unwanted and unacceptable side effects. Accordingly, the present invention furthermore provides medicaments comprising at least one of the compounds according to the invention and one or more further active compounds, in particular for treatment and/or prevention of the abovementioned diseases.

For example, the compounds of the present invention can be combined with known antihyperproliferative, cytostatic or cytotoxic substances for treatment of cancer disorders. The combination of the compounds according to the invention with other substances customary for cancer therapy or else with radiotherapy is indicated in particular.

Suitable active compounds for combinations which may be mentioned by way of example are:

Abraxane, afinitor, aldesleukin, alendronic acid, alfaferone, alitretinoin, allopurinol, aloprim, Aloxi, altretamine, aminoglutethimide, amifostine, amrubicin, amsacrine, anastrozole, anzmet, Aranesp, arglabin, arsenic trioxide, aromasin, 5-azacytidine, azathioprine, BCG or tice-BCG, bestatin, beta-methasone acetate, betamethasone sodium phosphate, bexarotene, bleomycin sulfate, broxuridine, bortezomib, busulfan, calcitonin, Campath, capecitabine, carboplatin, Casodex, cefesone, celmoleukin, cerubidin, chlorambucil, cisplatin, cladribin, clodronic acid, cyclophosphamide, cytarabine, dacarba-zine, dactinomycin, DaunoXome, Decadron, Decadron phosphate, delestrogen, denileukin diftitox, depomedrol, deslorelin, dexrazoxane, diethylstilbestrol, Diflucan, docetaxel, doxifluridine, doxo-rubicin, dronabinol, DW-166HC, Eligard, Elitek, Ellence, Emend, epirubicin, epoetin-alfa, Epogen, eptaplatin, ergamisol, Estrace, estradiol, estramustine sodium phosphate, ethynylestradiol, Ethyol, etidronic acid, Etopophos, etoposide, fadrozole, farstone, filgrastim, finasteride, fligrastim, floxuridine, fluconazole, fludarabin, 5-fluorodeoxyuridine monophosphate, 5-fluoruracil (5-FU), fluoxymesterone, flutamide, formestane, fosteabine, fotemustine, fulvestrant, Gammagard, gemcitabine, gemtuzumab, Gleevec, Gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-2β, interferon-alpha-nl, interferon-alpha-n3, interferon-beta, interferon-gamma-1α, interleukin-2, intron A, Iressa, irinotecan, kytril, lapatinib, lentinan sulfate, letrozole, leucovorin, leuprolide, leuprolide acetate, levamisole, levofolic acid calcium salt, levothroid, levoxyl, lomustine, lonidamine, Marinol, mechlorethamine, mecobalamin, medroxyprogesterone acetate, megestrol acetate, melphalan, Menest, 6-mercaptopurine, mesna, methotrexate, Metvix, miltefosine, minocycline, mitomycin C, mitotane, mitoxantrone, Modrenal, Myocet, nedaplatin, Neulasta, Neumega, Neupogen, nilutamide, Nolvadex, NSC-631570, OCT-43, octreotide, ondansetron hydrochloride, Orapred, oxaliplatin, paclitaxel, Pediapred, pegaspargase, Pegasys, pentostatin, Picibanil, pilocarpine hydrochloride, pirarubicin, plicamycin, porfimer sodium, prednimustine, prednisolone, prednisone, Premarin, procarbazine, Procrit, raltitrexed, RDEA119, Rebif, rhenium-186 etidronate, rituximab, roferon-A, romurtide, Salagen, sandostatin, sargramostim, semustine, sizofiran, sobuzoxane, Solu-Medrol, streptozocin, strontium-89 chloride, Synthroid, tamoxifen, tamsulosin, tasonermin, tastolactone, taxoter, teceleukin, temozolomide, teniposide, testosterone propionate, Testred, thioguanine, thiotepa, thyrotropin, tiludronic acid, topotecan, toremifen, tositumomab, tastuzumab, treosulfan, tretinoin, Trexall, trimethylmelamine, trimetrexate, triptorelin acetate, triptorelin pamoate, UFT, uridine, valrubicin, vesnarinone, vinblastine, vincristine, vindesine, vinorelbine, Virulizin, Zinecard, zinostatin-stimalamer, Zofran; ABI-007, acolbifen, Actimmune, Affinitak, aminopterin, arzoxifen, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, Osidem, paclitaxel polyglutamate, pamidronate disodium, PN-401, QS-21, quazepam, R-1549, raloxifen, ranpirnas, 13-cis-retinoic acid, satraplatin, seocalcitol, T-138067, Tarceva, taxoprexin, thymosin-alpha-1, tiazofurin, tipifarnib, tira-pazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example—without this list being conclusive:

Abraxane, aminoglutethimide, L-asparaginase, azathioprine, 5-azacytidine, bleomycin, busulfan, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenin, ethynylestradiol, etoposide, fludarabin phosphate, 5-fluorodeoxyuridine, 5-fluorodeoxyuridine mono-phosphate, 5-fluorouracil, fluoxymesterone, flutamide, hexamethylmelamine, hydroxyurea, hydroxy-progesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphonoacetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, tri-methylmelamine, uridine, vinblastine, vincristine, vindesine and vinorelbine.

The compounds according to the invention can also be combined in a very promising manner with biological therapeutics, such as antibodies (e.g. Avastin, Rituxan, Erbitux, Herceptin, cetuximab) and recombinant proteins.

The compounds according to the invention may also achieve positive effects in combination with other therapies directed against angiogenesis, such as, for example, with avastin, axitinib, regorafenib, recentin, sorafenib or sunitinib. Combinations with inhibitors of the proteasome and of mTOR and antihormones and steroidal metabolic enzyme inhibitors are particularly suitable because of their favorable profile of side effects.

Generally, the following aims can be pursued with the combination of compounds of the present invention with other agents having a cytostatic or cytotoxic action:
an improved activity in slowing down the growth of a tumor, in reducing its size or even in its complete elimination compared with treatment with an individual active compound;
the possibility of employing the chemotherapeutics used in a lower dosage than in monotherapy;
the possibility of a more tolerable therapy with few side effects compared with individual administration;
the possibility of treatment of a broader spectrum of tumor diseases;
achievement of a higher rate of response to the therapy;
a longer survival time of the patient compared with present-day standard therapy.

The compounds according to the invention can moreover also be employed in combination with radiotherapy and/or surgical intervention.

### Preparation of the Compounds According to the Invention

The preparation of the compounds according to the invention is comprehensively described in PCT/EP2009/007247, the disclosure of which is referred to in the present application and which is incorporated into this application by reference.

### Principles of the Preparation

### Preparation of the Compounds of the Formula (Ia) (4-O Derivatives)

The compounds according to the invention can be prepared by a process which is characterized by the following steps:
a) oxidation of a compound of the formula (IVd) to give the sulfoxide of the formula (IVc)
b₁) direct imination of the sulfoxide of the formula (IVc) to give a protected sulfoximine of the

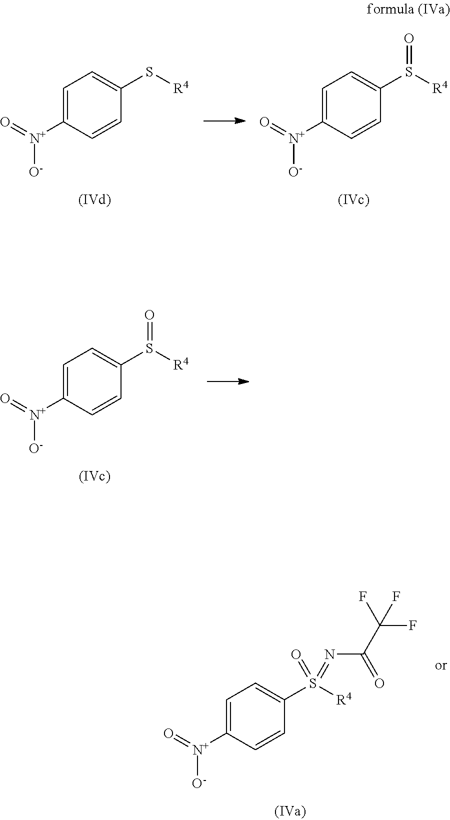

b2) imination of the sulfoxide of the formula (IVc) to give an unprotected sulfoximine of the formula (IVb) and subsequent introduction of the protective group to give a compound of the formula (IVa)

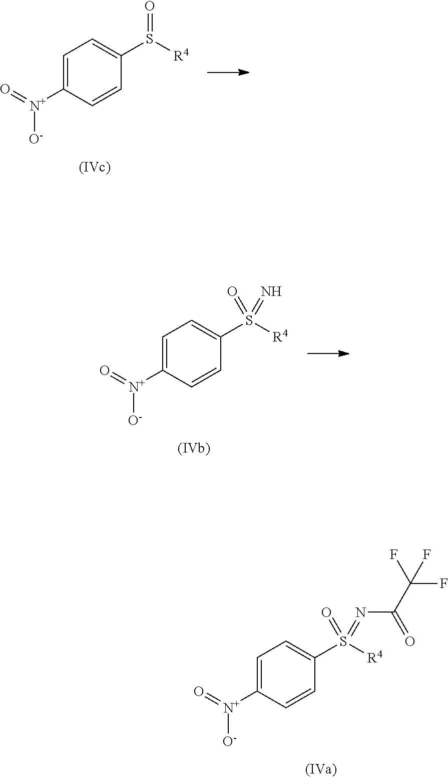

c) reduction of the compound of the formula (IVa) to give a compound of the formula (IV)

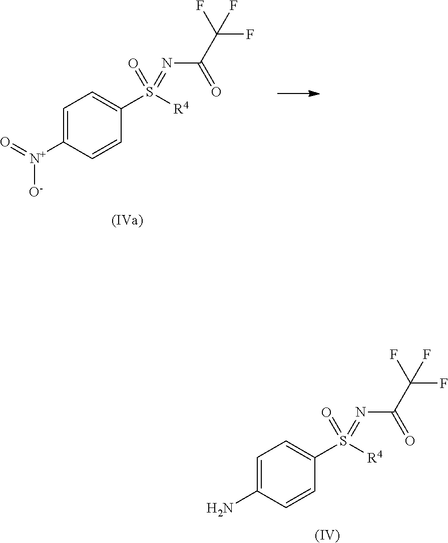

d) functionalization of the 4-position of 2,4-dichloro-5-iodopyrimidine (VII) by reaction with a mono-protected (PG=protective group) diol of the formula (VI) with formation of an intermediate of the formula (Va)

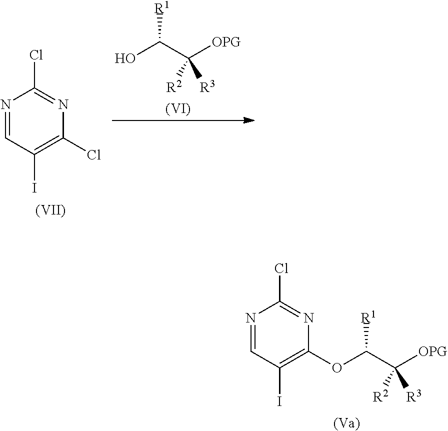

e) preparation of the 5-CF₃ intermediate (V)

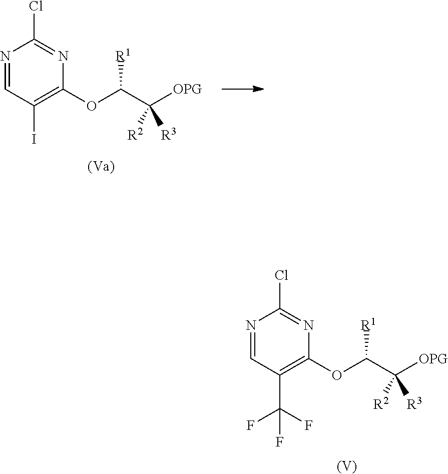

f) coupling of the compounds of the formulae (IV) and (V) to give the intermediate of the formula (III)

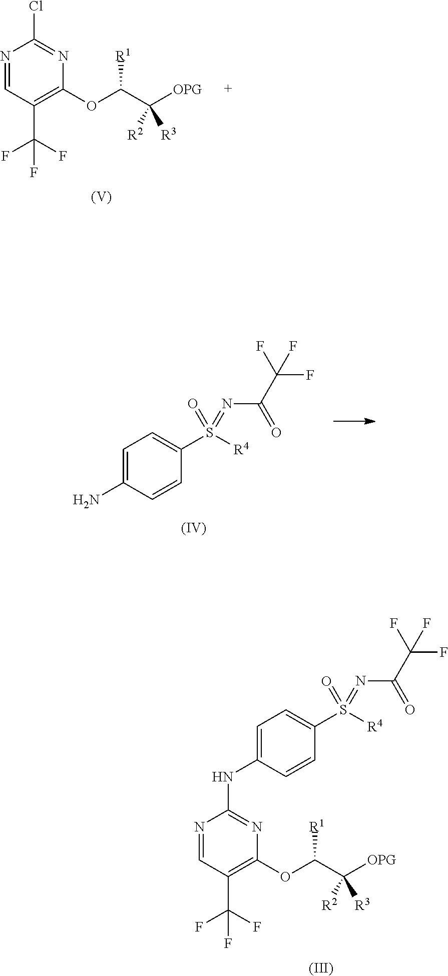

g) removal of the protective group (PG) with formation of (II)

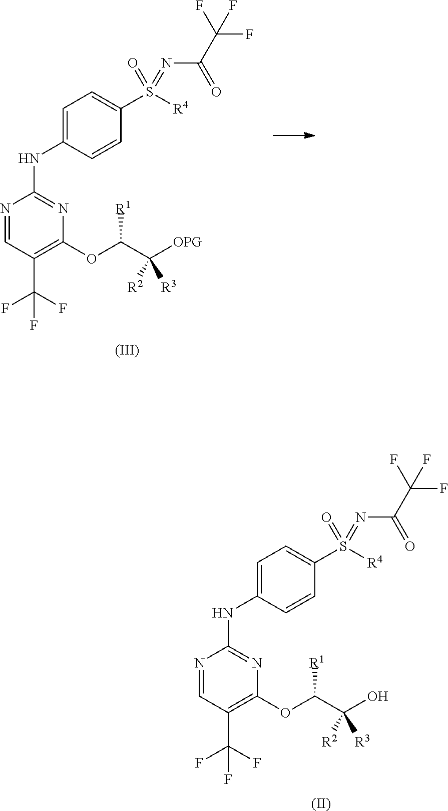

h) removal of the protective group on the sulfoximine with formation of (Ia)

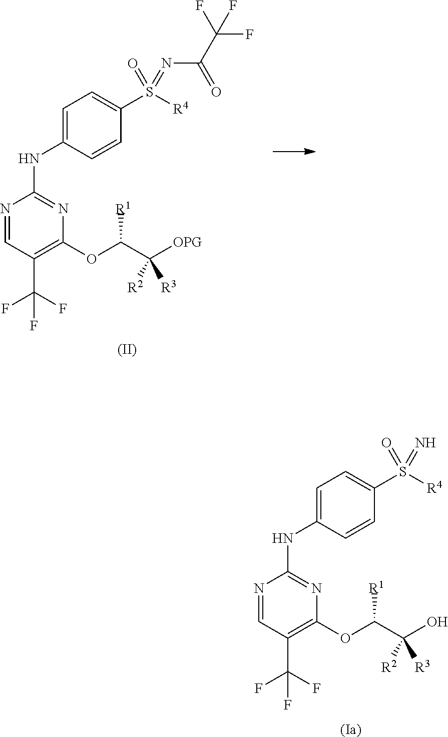

where the substituents R², R³ and R⁴ have the meanings given in the general formula (I).

### Preparation of the Compounds of the General Formula (Ib) (4-N Derivatives)

The compounds according to the invention can be prepared by a process which is characterized by the following steps:
a) oxidation of a compound of the formula (IVd) to give the sulfoxide of the formula (IVc)

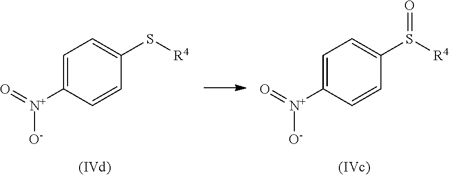

b₁) direct imination of the sulfoxide of the formula (IVc) to give a protected sulfoximine of the formula (IVa)

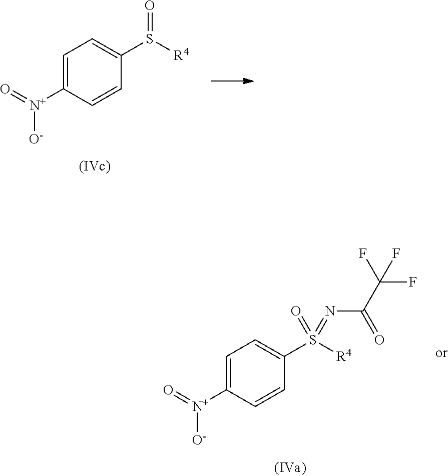

b2) imination of the sulfoxide of the formula (IVc) to give an unprotected sulfoximine of the formula (IVb) and subsequent introduction of the protective group to give a compound of the formula (IVa)

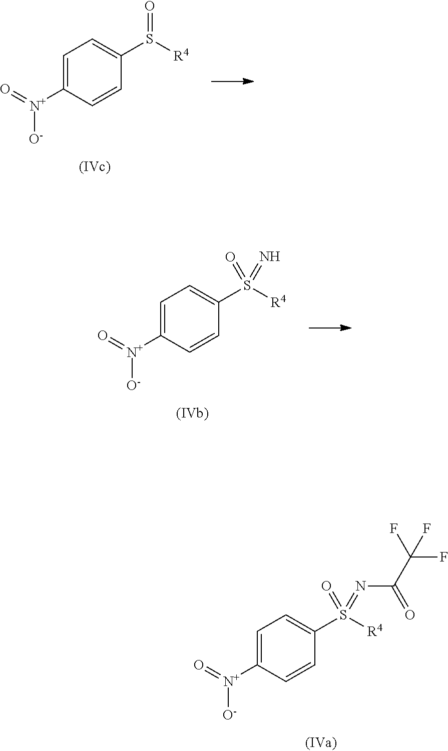

c) reduction of the compound of the formula (IVa) to give a compound of the formula (IV)

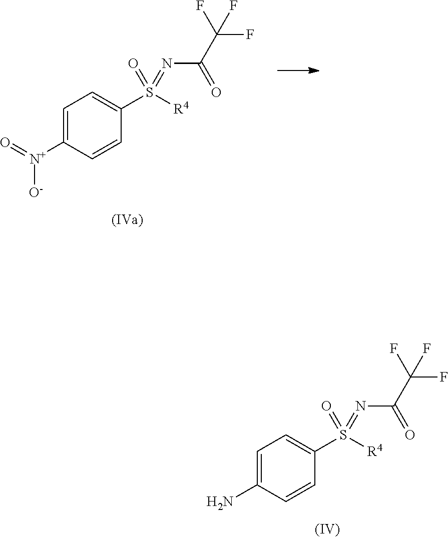

d) functionalization of the 4-position of 2,4-dichloro-5-trifluoromethylpyrimidine (VIIb) by reaction of an amine of the formula (VIa) with formation of an intermediate of the formula (Vb)

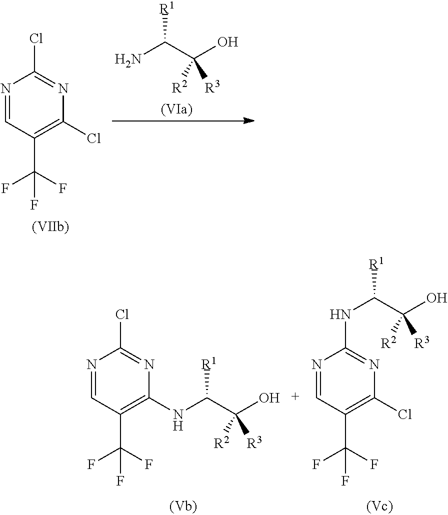

e) coupling of the compounds of the formulae (Vb) and (IV) to give the intermediate of the formula (IIb)

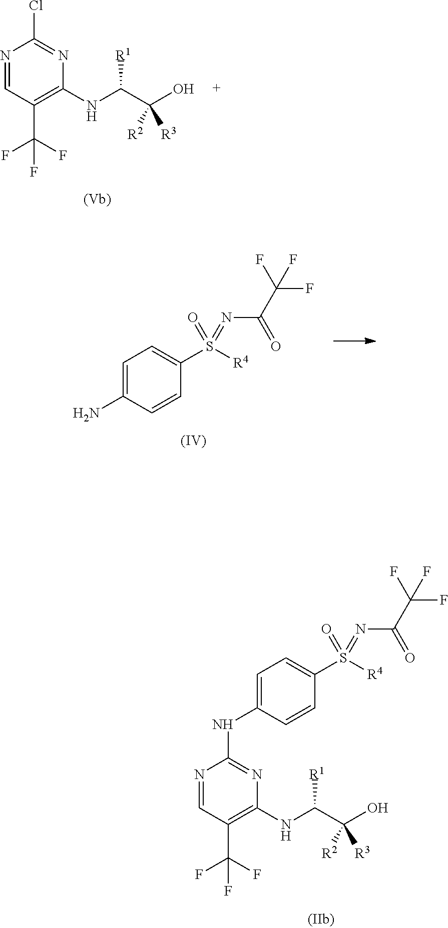

f) removal of the protective group on the sulfoximine with formation of (Ib)

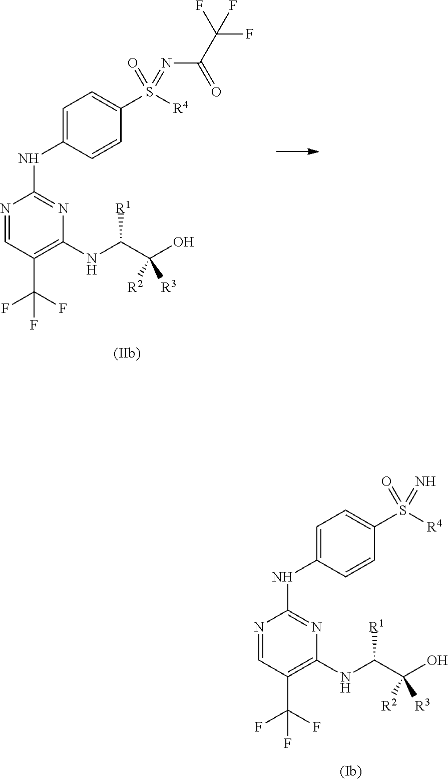

where the substituents R¹, R², R³ and R⁴ have the meanings given in the general formula (I).

### EXAMPLE 1

### (RS)—S-Cyclopropyl-S-(4-{[4-{[(1R,2R)-2-hydroxy-1-methylpropyl]oxy}-5-(trifluoromethyl)-pyrimidin-2-yl]amino}phenyl)sulfoximide

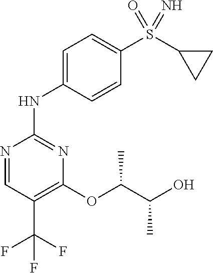 [SMILES: C[C@@H](O)[C@@H](C)Oc1nc(Nc2ccc(S(=N)(=O)C3CC3)cc2)ncc1C(F)(F)F]

The preparation of Example 1 is carried out according to Example 1 of PCT/EP2009/007247.

The diastereomer mixture was separated by preparative HPLC into the pure stereoisomers:

column: Chiralpak IA 5μ 250×30 mm
mobile phases: hexane/ethanol 8:2
flow rate: 40.0 ml/min
detector: UV 254 nm
temperature: room temperature
retention time: 10.8-13.4 min; stereoisomer 1 (=Example 1-SI-1)
13.6-18.5 min; stereoisomer 2 (=Example 1-SI-2)

### EXAMPLE 2

### (RS)—S-(4-{[4-{[(1R,2R)-2-Hydroxy-1-methylpropyl]oxy}-5-(trifluoromethyl)pyrimidin-2-yl]-amino}phenyl)-S-methylsulfoximide

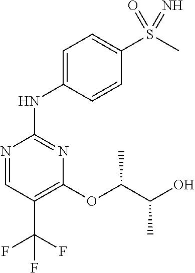 [SMILES: C[C@@H](O)[C@@H](C)Oc1nc(Nc2ccc(S(C)(=N)=O)cc2)ncc1C(F)(F)F]

The preparation of Example 2 is carried out according to Example 2 of PCT/EP2009/007247.

The diastereomer mixture was separated by preparative HPLC into the pure stereoisomers:

column: Chiralpak IC 5μ 250×20 mm
mobile phases: hexane/ethanol 8:2
buffer:hexane/0.1% DEA
flow rate: 25.0 ml/min
detector: UV 280 nm
temperature: room temperature
retention time: 9.5-12.1 min; stereoisomer 1 (=Example 2-SI-1)
13.1-16.0 min; stereoisomer 2 (=Example 2-SI-2)

### EXAMPLE 3

### (RS)—S-(4-{[4-{[(R)-2-Hydroxy-1,2-dimethylpropyl]oxy}-5-(trifluoromethyl)pyrimidin-2-yl]-amino}phenyl)-S-methylsulfoximide

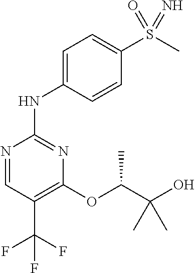 [SMILES: C[C@@H](Oc1nc(Nc2ccc(S(C)(=N)=O)cc2)ncc1C(F)(F)F)C(C)(C)O]

The preparation of Example 3 is carried out according to Example 3 of PCT/EP2009/007247.

The residue was purified by HPLC. This gave 31 mg (0.07 mmol; yield: 14%) of the product.

### EXAMPLE 4

### (RS)—S-Cyclopropyl-S-(4-{[4-{[(1R,2R)-2-hydroxy-1-methylpropyl]amino}-5-(trifluoromethyl)-pyrimidin-2-yl]amino}phenyl)sulfoximide

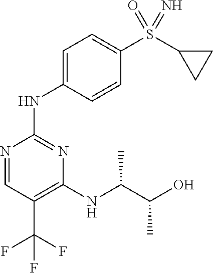 [SMILES: C[C@@H](O)[C@@H](C)Nc1nc(Nc2ccc(S(=N)(=O)C3CC3)cc2)ncc1C(F)(F)F]

The preparation of Example 4 is carried out according to Example 4 of PCT/EP2009/007247.

The diastereomer mixture was separated by preparative HPLC into the pure stereoisomers:

column: Chiralpak IA 5μ 250×20 mm
mobile phases: hexane/2-propanol 50:50
buffer:hexane/0.1% DEA
flow rate: 15.0 ml/min
detector: UV 254 nm
temperature: room temperature
retention time: 5.9-6.6 min; stereoisomer 1 (=Example 4-SI-1)
7.1-8.8 min; stereoisomer 2 (=Example 4-SI-2)

### EXAMPLE 5

### (RS)—S-Cyclopropyl-S-(4-{[4-{[(R)-2-hydroxy-1,2-dimethylpropyl]amino}-5-(trifluoromethyl)-pyrimidin-2-yl]amino}phenyl)sulfoximide

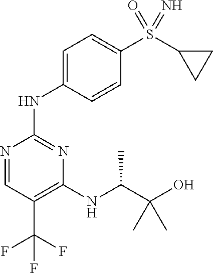 [SMILES: C[C@@H](Nc1nc(Nc2ccc(S(=N)(=O)C3CC3)cc2)ncc1C(F)(F)F)C(C)(C)O]

The preparation of Example 5 is carried out according to Example 5 of PCT/EP2009/007247.

The diastereomer mixture was separated by preparative HPLC into the pure stereoisomers:

column: Chiralpak AD-H 5μ 250×20 mm
mobile phases: hexane/2-propanol 60:40
buffer:hexane/0.1% DEA
flow rate: 20.0 ml/min
detector: UV 280 nm
temperature: room temperature
retention time: 5.1-6.3 min; stereoisomer 1 (=Example 5-SI-1)
8.0-10.8 min; stereoisomer 2 (=Example 5-SI-2)

### EXAMPLE 6

### (RS)—S-Ethyl-S-(4-{[4-{[(1R,2R)-2-hydroxy-1-methylpropyl]amino}-5-(trifluoromethyl)-pyrimidin-2-yl]amino}phenyl)sulfoximide

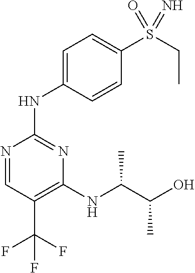 [SMILES: CCS(=N)(=O)c1ccc(Nc2ncc(C(F)(F)F)c(N[C@H](C)[C@@H](C)O)n2)cc1]

The preparation of Example 6 is carried out according to Example 6 of PCT/EP2009/007247.

The diastereomer mixture was separated by preparative HPLC into the pure stereoisomers:

column: Chiralpak AD-H 5μ 250×20 mm
mobile phases: hexane/2-propanol 60:40
buffer:hexane/0.1% DEA
flow rate: 20.0 ml/min
detector: UV 280 nm
temperature: room temperature
retention time: 6.2-6.8 min; stereoisomer 1 (=Example 6-SI-1)
7.2-8.9 min; stereoisomer 2 (=Example 6-SI-2)

### EXAMPLE 7

### (RS)—S-Ethyl-S-(4-{[4-{[(R)-2-hydroxy-1,2-dimethylpropyl]amino}-5-(trifluoromethyl)pyrimidin-2-yl]amino}phenyl)sulfoximide

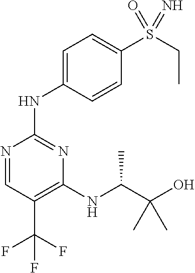 [SMILES: CCS(=N)(=O)c1ccc(Nc2ncc(C(F)(F)F)c(N[C@H](C)C(C)(C)O)n2)cc1]

The preparation of Example 7 is carried out according to Example 7 of PCT/EP2009/007247.

The diastereomer mixture was separated by preparative HPLC into the pure stereoisomers:

column: Chiralpak AD-H 5μ 250×20 mm
mobile phases: A:hexane B:2-propanol
buffer: hexane/0.1% DEA
gradient: 20->40% B(20′)+40% B(5′)
flow rate: 10.0 ml/min
detector: UV 280 nm
temperature: room temperature
retention time: 17.5-19.8 min; stereoisomer 1 (=Example 7-SI-1)
20.1-22.0 min; stereoisomer 2 (=Example 7-SI-2)

### EXAMPLE 8

### (RS)—S-(4-{[4-{[(1R,2R)-2-Hydroxy-1-methylpropyl]amino}-5-(trifluoromethyl)pyrimidin-2-yl]-amino}phenyl)-S-methylsulfoximide

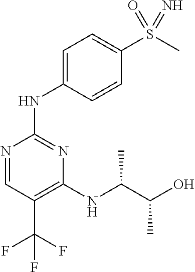 [SMILES: C[C@@H](O)[C@@H](C)Nc1nc(Nc2ccc(S(C)(=N)=O)cc2)ncc1C(F)(F)F]

The preparation of Example 8 is carried out according to Example 8 of PCT/EP2009/007247.

The diastereomer mixture was separated by preparative HPLC into the pure stereoisomers:

column: Chiralpak IC 5μ 250×20 mm
mobile phases: hexane/ethanol 50:50
buffer:hexane/0.1% DEA
flow rate: 20.0 ml/min
detector: UV 254 nm
temperature: room temperature
retention time: 5.1-5.8 min; stereoisomer 1 (=Example 8-SI-1)
6.1-6.7 min; stereoisomer 2 (=Example 8-SI-2)

### EXAMPLE 9

### (RS)—S-(4-{[4-{[(1R)-2-Hydroxy-1,2-dimethylpropyl]amino}-5-(trifluoromethyl)pyrimidin-2-yl]amino}phenyl)-S-methylsulfoximide

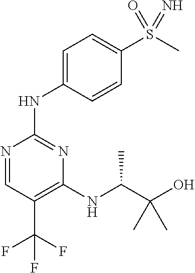 [SMILES: C[C@@H](Nc1nc(Nc2ccc(S(C)(=N)=O)cc2)ncc1C(F)(F)F)C(C)(C)O]

The preparation of Example 9 is carried out according to Example 9 of PCT/EP2009/007247.

The diastereomer mixture was separated by preparative HPLC into the pure stereoisomers:

column: Chiralpak IC 5μ 250×20 mm
mobile phases: hexane/ethanol 80:20
flow rate: 30.0 ml/min
detector: UV 254 nm
temperature: room temperature
retention time: 6.0-6.7 min; stereoisomer 1 (=Example 9-SI-1)
7.1-8.9 min; stereoisomer 2 (=Example 9-SI-2)

### EXAMPLE 10

### 10.1 Assay 1: CDK1/CycB Kinase Assay

Recombinant CDK1 and CycB-GST fusion proteins, purified from baculovirus-infected insect cells (Sf9), were purchased from ProQinase GmbH, Freiburg, Germany. The histon HIS used as kinase substrate is commercially available from Sigma.

CDK1/CycB (200 ng/measuring point) was incubated for 10 min at 22° C. in the presence of various concentrations of test substances (0 μM, and within the range 0.01-100 μM) in assay buffer [50 mM Tris/HCl pH8.0, 10 mM MgCl₂, 0.1 mM Na ortho-vanadate, 1.0 mM dithiothreitol, 0.5 μM adenosine triphosphate (ATP), 10 μg/measuring point histon IIIS, 0.2 μCi/measuring point ³³P-gamma ATP, 0.05% NP40, 1.25% dimethyl sulfoxide]. The reaction was stopped by adding EDTA solution (250 mM, pH 8.0, 15 μl/measuring point).

From each reaction mixture, 15 μl were applied to P30 filter strips (from Wallac), and unincorporated ³³P-ATP was removed by washing the filter strips three times, for 10 min each time, in 0.5% phosphoric acid. After drying the filter strips for 1 hour at 70° C., the filter strips were covered with scintillator strips (MeltiLex™ A, from Wallac) and stoved for 1 hour at 90° C. The amount of incorporated ³³P (substrate phosphorylation) was determined by scintillation measurement in a gamma-radiation measuring instrument (Wallac). The measured data were standardized to 0% inhibition (enzyme reaction without inhibitor) and 100% inhibition (all assay components except enzyme). The IC₅₀ values were determined by means of a 4-parameter fit using the company's own software.

### 10.2 Assay 2: CDK2/CycE Kinase Assay

Recombinant CDK2 and CycE-GST fusion proteins, purified from baculovirus-infected insect cells (Sf9), were purchased from ProQinase GmbH, Freiburg, Germany. Histon HIS, used as kinase substrate, was purchased from Sigma.

CDK2/CycE (50 ng/measuring point) was incubated for 10 min at 22° C. in the presence of various concentrations of test substances (0 μM, and within the range 0.01-100 μM) in assay buffer [50 mM Tris/HCl pH 8.0, 10 mM MgCl₂, 0.1 mM Na ortho-vanadate, 1.0 mM dithiothreitol, 0.5 μM adenosine triphosphate (ATP), 10 μg/measuring point histon IIIS, 0.2 μCi/measuring point ³³P-gamma ATP, 0.05% NP40, 1.25% dimethyl sulfoxide]. The reaction was stopped by adding EDTA solution (250 mM, pH 8.0, 15 μl/measuring point).

From each reaction mixture, 15 μl was applied to P30 filter strips (from Wallac), and unincorporated ³³P-ATP was removed by washing the filter strips three times, for 10 min each time, in 0.5% phosphoric acid. After drying the filter strips for 1 hour at 70° C., the filter strips were covered with scintillator strips (MeltiLex™ A, from Wallac) and stoved for 1 hour at 90° C. The amount of incorporated ³³P (substrate phosphorylation) was determined by scintillation measurement in a gamma-radiation measuring instrument (Wallac). The measured data were standardized to 0% inhibition (enzyme reaction without inhibitor) and 100% inhibition (all assay components except enzyme). The IC₅₀ values were determined by means of a 4-parameter fit using the company's own software.

### 10.3 Assay 3: VEGF Receptor-2 Kinase Assay

Recombinant VEGF receptor tyrosine kinase-2 was purified as GST fusion protein from baculovirus-infected insect cells (Sf9). Poly-(Glu4Tyr), used as kinase substrate, was purchased from Sigma.

VEGF receptor tyrosine kinase (90 ng/measuring point) was incubated for 10 min at 22° C. in the presence of various concentrations of test substances (0 μM, and within the range 0.001-30 μM) in 30 μl assay buffer [40 mM Tris/HCl pH5.5, 10 mM MgCl₂, 1 mM MnCl₂, 3 μM Na ortho-vanadate, 1.0 mM dithiothreitol, 8 μM adenosine triphosphate (ATP), 0.96 μg/measuring point poly-(Glu4Tyr), 0.2 μCi/measuring point ³³P-gamma ATP, 1.4% dimethyl sulfoxide]. The reaction was stopped by adding EDTA solution (250 mM, pH 8.0, 15 μl/measuring point).

From each reaction mixture, 15 μl was applied to P30 filter strips (from Wallac), and unincorporated ³³P-ATP was removed by washing the filter strips three times, for 10 min each time, in 0.5% phosphoric acid. After drying the filter strips for 1 hour at 70° C., the filter strips were covered with scintillator strips (MeltiLex™ A, from Wallac) and stoved for 1 hour at 90° C. The amount of incorporated ³³P (substrate phosphorylation) was determined by scintillation measurement in a gamma-radiation measuring instrument (Wallac). The measured data were standardized to 0% inhibition (enzyme reaction without inhibitor) and 100% inhibition (all assay components except enzyme). The IC₅₀ values were determined by means of a 4-parameter fit using the company's own software.

### 10.4 Assay 4: Proliferation Assay

### EXAMPLE 1

### Proliferation Assay

Cultivated human tumor cells (originally obtained from ATCC, HeLa-MaTu and HeLa-MaTu-ADR, originally obtained from Epo GmbH, Berlin, Germany) were plated at a density of 1000 to 5000 cells/measuring point, depending on the growth rate of the cell line, in a 96-well multititer plate in 200 μl of growth medium (DMEM/HAMS F12, 2 mM L-glutamine, 10% fetal calf serum). After 24 hours the cells of one plate (zero-point plate) were stained with crystal violet (see below), whereas the medium of the other plates was replaced with fresh culture medium (200 μl), to which the test substances had been added at various concentrations (0 μM, and in the range 0.01-30 μM; the final concentration of the solvent dimethyl sulfoxide was 0.5%). The cells were incubated for 4 days in the presence of the test substances. Cellular proliferation was determined by staining the cells with crystal violet: the cells were fixed by adding 20 μl/measuring point of an 11% glutaraldehyde solution for 15 min at room temperature. After washing the fixed cells with water three times, the plates were dried at room temperature. The cells were stained by adding 100 μl/measuring point of a 0.1% crystal violet solution (pH adjusted to pH3 by adding acetic acid). After washing the stained cells with water three times, the plates were dried at room temperature. The dye was dissolved by adding 100 μl/measuring point of a 10% acetic acid solution. The extinction was determined photometrically at a wavelength of 595 nm. The percentage change in cell growth was calculated by standardization of the measured values to the extinction values of the zero-point plate (=0%) and the extinction of the untreated (0 μM) cells (=100%). The measured data were standardized to 0% inhibition (cell proliferation without inhibitor) and 100% inhibition (zero-point plate). The IC₅₀ values were determined by means of a 4-parameter fit using the company's own software.

The substances were examined in the following cell lines which, in an exemplary manner, represent the stated indications:

TABLE 1

| Tumor indication | Cell line |
|---|---|
| estrogen receptor-negative mamma carcinoma | SK-BR-3 |
| | MDA-MB 231 |
| | MDA-MB 453 |
| estrogen receptor-positive mamma carcinoma | MCF7 |
| ovarial carcinoma | OVCAR-8 |
| | NCI-ADR-Res |
| | A2780 |
| | A2780-Cis |
| colon/rectum carcinoma | HT29 |
| | Caco-2 |
| | SW480 |
| | HCT116 |
| prostate carcinoma | DU145 |
| | PC3 |
| non-small-cell bronchial carcinoma | NCI-H460 |
| | A549 |
| | H1975 |
| small-cell bronchial carcinoma | NCI-H69 |
| kidney carcinoma | Caki2 |
| | 786-0 |
| pancreas carcinoma | MIA PaCa-2 |
| cervix carcinoma | HeLa |
| | HeLa-MaTu |
| | HeLa-MaTu-ADR |
| skin: epidermis | A431 |
| skin: melanoma | A375 |
| leukemia | MOLM-13 |

### 10.5 In-Vivo-Models

Tumor cells grown in cell culture were implanted subcutaneously in the flank of female or male NMRI nude mice. The treatment was started as soon as the tumors had grown to a size of approx. 20 mm² The study was ended as soon as the tumors in one of the groups reached a size of approx. 150 mm².

The following test groups were used:

Vehicle group: treatment with solubilizer (40% PEG400/60% water)

Treatment groups: specified under 10.8.

The studies were designed for determining the initial response of the human tumor model to the treatment with the Exemplary compound 2-SI-2. Tumor growth inhibition in percent (TGI) was either calculated at at the end of the studies from the tumor weights (TGI_{TW}) using the formula 100×[1−(tumor weight of the treatment group/tumor weight of the vehicle group)], or on the day the vehicle group had to be ended from the tumor areas (TGI_{TA}) using the formula 100×[1−(tumor area of the treatment group on the day of the measurement−tumor area of the treatment group prior to treatment)/(tumor area of the vehicle group on the day of the measurement−tumor area of the vehicle group prior to treatment)]. In the case of a tumor growth inhibition of more than 50%, the treatment was considered to have been effective.

The Exemplary compound 2-SI-2 was examined in the following in vivo tumor models which, in an exemplary manner, represent the stated indications:

TABLE 2

| Tumor indication | in vivo tumor model |
|---|---|
| estrogen receptor-negative mamma carcinoma | MDA-MB 231 |
| ovarial carcinoma | A2780Cis |
| colon/rectum carcinoma | HCT116 |
| small-cell bronchial carcinoma | NCI-H69 |
| | NCI-H146 |
| | NCI-H526 |
| | NCI-H82 |
| cervix carcinoma | HeLa-MaTu |
| | HeLa-MaTu-ADR |

### 10.6 Results of the Enzyme Assays

TABLE 3

| | Enzyme | | |
|---|---|---|---|
| | CDK1/CycB | CDK2/CycE | VEGF-R2 |
| | (Assay 1) | (Assay 2) | (Assay 3) |
| | Concentration for half-maximal inhibition of the | | |
| Ex. | enzyme activity or cell proliferation, IC₅₀ [nM] | | |
| 1-SI-1 | 9 | 7 | 114 |
| 1-SI-2 | 7 | 9 | 163 |
| 2-SI-1 | 5 | 6 | 84 |
| 2-SI-2 | 4 | 5 | 281 |
| 3 | 13 | 10 | |
| 4-SI-1 | 6 | 6 | 46 |
| 4-SI-2 | | | |
| 5-SI-1 | 25 | 8 | 70 |
| 5-SI-2 | 9 | 8 | 82 |
| 6-SI-1 | 10 | 5 | 73 |
| 6-SI-2 | 5 | 5 | 71 |
| 7-SI-1 | 24 | 4 | 143 |
| 7-SI-2 | 7 | 5 | 136 |
| 8-SI-1 | 11 | 6 | 116 |
| 8-SI-2 | 3 | 4 | 81 |
| 9-SI-1 | 4 | 5 | 158 |
| 9-SI-2 | 17 | 3 | 154 |

### 10.7 Results of the Proliferation Assay

TABLE 4

| | IC50 [nM] | | | | | | | | | | | | | | | |
|---|---|---|---|---|---|---|---|---|---|---|---|---|---|---|---|---|
| Ex. | 1 | | 2 | | 3 | 4 | 5 | | 6 | | 7 | | 8 | | 9 | |
| Cell line | SI-1 | SI-2 | SI-1 | SI-2 | | SI-1 | SI-1 | SI-2 | SI-1 | SI-2 | SI-1 | SI-2 | SI-1 | SI-2 | SI-1 | SI-2 |
| SK-BR-3 | | 13 | | 21 | | 33 | | | | | | | | | | |
| MDA-MB | | 18 | | 16 | | | | | | | | | | | | |
| 231 | | | | | | | | | | | | | | | | |
| MDA-MB | | 15 | | 16 | | 11 | | | | | | | | | | |
| 453 | | | | | | | | | | | | | | | | |
| MCF7 | 48 | 15 | 39 | 11 | 70 | 34 | 33 | 34 | 59 | 26 | 106 | 39 | 44 | 23 | 89 | 271 |
| OVCAR-8 | | 12 | | 14 | | | | | | | | | | | | |
| NCI-ADR- | | 33 | | 29 | | 37 | | | | | | | | | | |
| Res | | | | | | | | | | | | | | | | |
| A2780 | | | | 3 | | | | | | | | | | | | |
| A2780-Cis | | | | 10 | | | | | | | | | | | | |
| HT29 | | 29 | | 28 | | 12 | | | | | | | | | | |
| Caco-2 | 80 | 16 | 80 | 24 | 98 | 35 | 112 | 42 | 120 | 26 | 196 | 67 | 32 | 41 | 61 | 200 |
| SW480 | | 15 | | 19 | | 22 | | | | | | 45 | | 31 | | |
| HCT116 | | 18 | | 16 | | 20 | | 28 | | 24 | | 17 | | 17 | | |
| DU145 | 45 | 8 | 28 | 9 | 76 | 31 | 107 | 26 | 100 | 197 | 152 | 42 | 64 | 30 | 34 | 185 |
| PC3 | | 25 | | 27 | | <10 | | | | | | | | | | |
| NCI-H460 | 48 | 12 | 68 | 16 | 113 | 16 | 70 | 33 | 50 | 24 | 119 | 51 | 30 | 28 | 34 | 112 |
| A549 | | 20 | | 23 | | <10 | | | | | | | | | | |
| H1975 | | 11 | | 14 | | 16 | | | | | | | | | | |
| NCI-H69 | | 37 | | 37 | | | | | | | | | | | | |
| Caki2 | | 26 | | 24 | | <10 | | | | | | | | | | |
| 786-O | | 20 | | 22 | | 30 | | | | | | | | | | |
| MIA PaCa-2 | | 21 | | 19 | | 14 | | | | | | | | | | |
| HeLa | | 12 | | 13 | | 33 | | 50 | | 32 | | 33 | | 25 | | |
| HeLa-MaTu | 13 | 11 | 12 | 8 | 70 | 10 | 22 | 10 | 16 | 10 | 27 | 10 | 14 | 10 | 20 | 21 |
| HeLa-MaTu- | 35 | 8 | 32 | 7 | 63 | 16 | 63 | 24 | 35 | 18 | 112 | 36 | 30 | 24 | 19 | 114 |
| ADR | | | | | | | | | | | | | | | | |
| A431 | | 14 | | 17 | | 20 | | | | | | | | | | |
| A375 | | 14 | | 15 | | | | | | | | | | | | |
| MOLM-13 | | 14 | | 9 | | | | | | | | | | | | |

The results of the proliferation assays demonstrate the efficacy of the exemplary compounds in a large number of different human tumor cells, with a pronounced uniform profile. These data indicate broad applicability of the exemplary compounds for the treatment of solid as well as haematological tumor disorders of various histological types.

### 10.8 Results of the In Vivo-Tumor Models

### 10.8.1 Cervix Carcinoma Model

### Study:

Efficacy in the HeLa-MaTu human cervix carcinoma xenograft model

### Test System:

HeLa-MaTu human cervix carcinoma cells implanted into female NMRI nude mice

### Administration Form:

oral (stomach tube)

### Formulation

a) 0.05 mg/ml, 0.10 mg/ml, 0.15 mg/ml, 0.2 mg/ml of Example 2-SI-2 in 40% (v/v) PEG 400 in water
b) 0.15 mg/ml, 0.2 mg/ml, 0.25 mg/ml of Example 2-SI-2 in 40% (v/v) PEG 400 in water

### Dosage and Treatment Protocol:

a) 0.5 to 2.0 mg/kg (1.5 to 6.0 mg/m²) 1× per day
   b) 1.5 to 2.5 mg/kg (4.5 to 7.5 mg/m²) 2× per day on 2 successive days, followed by 5 treatment-free days

### Significant Results

a) TGI_{TW}: 97% at 2.0 mg/kg
b) TGI_{TW}: 98% at 2.5 mg/kg, signs of tumor regression

### 10.8.2 Multi-Drug-Resistant Cervix Carcinoma Model

### Study:

Efficacy in the HeLa-MaTu-ADR Res. xenograft model

### Test System:

HeLa-MaTu-ADR multidrug-resistant human cervix carcinoma cells implanted into female NMRI nude mice.

### Administration Form:

oral (stomach tube)

Formulation
a) 0.15 mg/ml, 0.20 mg/ml of Example 2-SI-2 in 40% (v/v) PEG 400 in water
b) 0.20 mg/ml, 0.25 mg/ml of Example 2-SI-2 in 40% (v/v) PEG 400 in water

### Dosage and Treatment Protocol:

a) 1.5 and 2.0 mg/kg (4.5 and 6.0 mg/m²), 1× per day
b) 2.0 and 2.5 mg/kg (6.0 and 7.5 mg/m²), 2× per day on 2 successive days, followed by 5 treatment-free days

### Significant Results

a) TGI_{TW}: 97% at 2.0 mg/kg
b) TGI_{TW}: 90% at 2.5 mg/kg, signs of tumor regression

### 10.8.3 Colon Carcinoma Model

### Study:

Efficacy in the HCT116 human colorectal xenograft model.

### Test System:

HCT116 human colorectal tumor cells implanted into female NMRI nude mice.

### Administration Form:

oral (stomach tube).

### Formulation

a) 0.15 mg/ml, 0.20 mg/ml of Example 2-SI-2 in 40% (v/v) PEG 400 in water
b) 0.20 mg/ml, 0.25 mg/ml of Example 2-SI-2 in 40% (v/v) PEG 400 in water
c) 0.40 mg/ml, 0.50 mg/ml, 0.60 mg/ml of Example 2-SI-2 in 40% (v/v) PEG 400 in water

### Dosage and Treatment Protocol:

a) 1.5 and 2.0 mg/kg (4.5 and 6.0 mg/m²), 1× per day.
b) 2.0 and 2.5 mg/kg (6.0 and 7.5 mg/m²), 2× per day on 2 successive days, followed by 5 treatment-free days.
c) 4.0 to 6.0 mg/kg (12 to 18 mg/m²), 1× per day on 2 successive days, followed by 5 treatment-free days.

### Significant Results

a) TGI_{TW}: 67% at 2.0 mg/kg.
b) TGI_{TW}: 57% at 2.5 mg/kg, signs of tumor regression.
c) TGI_{TW}: 83% at 5.0 mg/kg.

### 10.8.4 Small-Cell Lung Carcinoma Model

### Study:

Efficacy in the NCI-H69 human small-cell lung tumor model.

### Test System:

NCI-H69 human small-cell lung tumor cells implanted into female NMRI nude mice.

### Administration Form:

oral (stomach tube).

### Formulation

a) 0.20 mg/ml of Example 2-SI-2 in 40% (v/v) PEG 400 in water
b) 0.25 mg/ml of Example 2-SI-2 in 40% (v/v) PEG 400 in water

### Dosage and Treatment Protocol:

a) 2.0 mg/kg (6.0 mg/m²), 1× per day.
b) 2.5 mg/kg (7.5 mg/m²), 2× per day on 2 successive days, followed by 5 treatment-free days.

### Significant Results

a) TGI_{TA} (measured on the day the vehicle group was ended): 99% at 2.0 mg/kg.
b) TGI_{TA}: 110% at 2.5 mg/kg

### 10.8.5 Small-Cell Lung Carcinoma Model

### Study:

Efficacy in the NCI-H146 human small-cell lung tumor model.

### Test System:

NCI-H146 human small-cell lung tumor cells implanted into female NMRI nude mice.

### Administration Form:

oral (stomach tube).

### Formulation

a) 0.20 mg/ml of Example 2-SI-2 in 40% (v/v) PEG 400 in water
b) 0.20 mg/ml of Example 2-SI-2 in 40% (v/v) PEG 400 in water

### Dosage and Treatment Protocol:

a) 2.0 mg/kg (6.0 mg/m²), 1× per day.
b) 2.0 mg/kg (6.0 mg/m²), 2× per day on 2 successive days, followed by 5 treatment-free days.

### Significant Results

a) TGI_{TW}: 95% at 2.0 mg/kg.
b) TGI_{TW}: 82% at 2.0 mg/kg

### 10.8.6 Small-Cell Lung Carcinoma Model

### Study:

Efficacy in the NCI-H82 human small-cell lung tumor model.

### Test System:

NCI-H82 human small-cell lung tumor cells implanted into female NMRI nude mice.

### Administration Form:

oral (stomach tube).

### Formulation

a) 0.17 mg/ml of Example 2-SI-2 in 40% (v/v) PEG 400 in water

### Dosage and Treatment Protocol:

a) 1.7 mg/kg (5.1 mg/m²), 2× per day on 3 successive days, followed by 4 treatment-free days.

### Significant Results

a) TGI_{TW}: 86% at 1.7 mg/kg.

### 10.8.7 Small-Cell Lung Carcinoma Model

### Study:

Efficacy in the NCI-H526 human small-cell lung tumor model.

### Test System:

NCI-H526 human small-cell lung tumor cells implanted into female NMRI nude mice.

### Administration Form:

oral (stomach tube).

### Formulation

a) 0.20 mg/ml of Example 2-SI-2 in 40% (v/v) PEG 400 in water
b) 0.20 mg/ml of Example 2-SI-2 in 40% (v/v) PEG 400 in water
c) 0.15 mg/ml of Example 2-SI-2 in 40% (v/v) PEG 400 in water
d) 0.17 mg/ml of Example 2-SI-2 in 40% (v/v) PEG 400 in water

### Dosage and Treatment Protocol:

a) 2.0 mg/kg (6.0 mg/m²), 1× per day.
b) 2.0 mg/kg (6.0 mg/m²), 2× per day on 2 successive days, followed by 5 treatment-free days.
c) 1.5 mg/kg (4.5 mg/m²), 2× per day on 3 successive days, followed by 4 treatment-free days.
d) 1.7 mg/kg (5.1 mg/m²), 2× per day on 3 successive days, followed by 4 treatment-free days.

### Significant Results

a) TGI_{TW}: 98% at 2.0 mg/kg.
b) TGI_{TW}: 72% at 2.0 mg/kg.
c) TGI_{TW}: 79% at 1.5 mg/kg.
d) TGI_{TW}: 88% at 1.7 mg/kg.

### 10.8.8 Breast Carcinoma Model

### Study:

Efficacy in the MDA-MB231 human breast tumor model MDA-MB231.

### Test System:

MDA-MB231 human breast tumor cells implanted into female NMRI nude mice.

### Administration Form:

oral (stomach tube).

### Formulation

a) 0.20 mg/ml of Example 2-SI-2 in 40% (v/v) PEG 400 in water
b) 0.25 mg/ml of Example 2-SI-2 in 40% (v/v) PEG 400 in water
c) 0.15 mg/ml of Example 2-SI-2 in 40% (v/v) PEG 400 in water
d) 0.17 mg/ml of Example 2-SI-2 in 40% (v/v) PEG 400 in water

### Dosage and Treatment Protocol:

a) 2.0 mg/kg (6.0 mg/m²), 1× per day.
b) 2.5 mg/kg (7.5 mg/m²), 2× per day on 2 successive days, followed by 5 treatment-free days.
c) 1.5 mg/kg (4.5 mg/m²), 2× per day on 3 successive days, followed by 4 treatment-free days.
d) 1.7 mg/kg (5.1 mg/m²), 2× per day on 3 successive days, followed by 4 treatment-free days.

### Significant Results

a) TGI_{TA} (measured on the day the vehicle group was ended): 92% at 2.0 mg/kg.
b) TGI_{TA}: 76% at 2.5 mg/kg.
c) TGI_{TA}: 70% at 1.5 mg/kg.
d) TGI_{TA}: 70% at 1.7 mg/kg.

### 10.8.9 Ovarial Carcinoma Model

### Study:

Efficacy in the A2780-Cis human ovarial tumor model.

### Test System:

Efficacy in the A2780-Cis human ovarial tumor model A2780-Cis cisplatin-resistant human ovarial tumor cells implanted into female NMRI nude mice.

### Administration Form:

oral (stomach tube).

### Formulation

a) 0.20 mg/ml of Example 2-SI-2 in 40% (v/v) PEG 400 in water
b) 0.15 mg/ml of Example 2-SI-2 in 40% (v/v) PEG 400 in water
c) 0.17 mg/ml of Example 2-SI-2 in 40% (v/v) PEG 400 in water

### Dosage and Treatment Protocol:

a) 2.0 mg/kg (6.0 mg/m²), 1× per day.
b) 1.5 mg/kg (4.5 mg/m²), 2× per day on 3 successive days, followed by 4 treatment-free days.
c) 1.7 mg/kg (5.1 mg/m²), 2× per day on 3 successive days, followed by 4 treatment-free days.

### Significant Results

a) TGI_{TW}: 85% at 2.0 mg/kg.
b) TGI_{TW}: 88% at 1.5 mg/kg.
c) TGI_{TW}: 92% at 1.7 mg/kg.

The results of the treatment study with the Exemplary compound 2-SI-2 in monotherapies confirm the tumor growth-inhibiting activity of the exemplary compound in animal models of human cervix tumors, small-cell bronchial tumors, colorectal tumors, breast tumors and ovarial tumors. The exemplary compound shows its efficacy in various administration protocols including administration once per day and several times per day, and comprising treatment-free intervals or managing without treatment-free intervals. Surprisingly, the compound is effective even in tumor models which respond poorly, if at all, to the treatment of cytostatic drugs approved for clinical use.

1. A method for the treatment of a tumor disorder comprising administering to a human in need thereof a compound of the general formula (I) 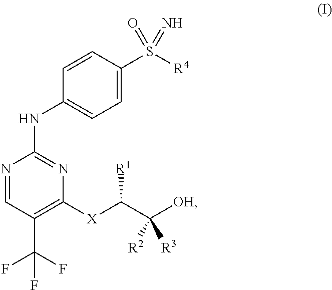
in which
X represents —O— or —NH—, and
R¹ represents a methyl, ethyl, propyl or isopropyl group, and
R² and R³ independently of one another represent hydrogen, a methyl or ethyl group, and
R⁴ represents a C₁-C₆-alkyl group or a C₃-C₇-cycloalkyl ring,
or a physiologically acceptable salt, diastereomer or enantiomer thereof.

2. The method according to claim 1, wherein
X represents —O— or —NH—, and
R¹ represents a methyl group, and
R² represents a methyl group, and
R³ represents hydrogen or a methyl group, and
R⁴ represents a methyl or ethyl group or represents a cyclopropyl ring.

3. The method according to claim 1, wherein the compound is selected from
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulfoximide
(RS)—S-(4-{[4-{[(1R,2R)-2-hydroxy-1-methylpropyl]oxy}-5-(trifluoromethyl)pyrimidin-2-yl]amino}phenyl)-S-methylsulfoximide
(RS)—S-(4-{[4-{[(R)-2-hydroxy-1,2-dimethylpropyl]oxy}-5-(trifluoromethyl)pyrimidin-2-yl]amino}phenyl)-S-methylsulfoximide
(RS)—S-cyclopropyl-S-(4-{[4-{[(1R,2R)-2-hydroxy-1-methylpropyl]amino}-5-(trifluoromethyl)pyrimidin-2-yl]amino}phenyl)sulfoximide
(RS)—S-cyclopropyl-S-(4-{[4-{[(R)-2-hydroxy-1,2-dimethylpropyl]amino}-5-(trifluoromethyl)pyrimidin-2-yl]amino}phenyl)sulfoximide
(RS)—S-ethyl-S-(4-{[4-{[(1R,2R)-2-hydroxy-1-methylpropyl]amino}-5-(trifluoromethyl)pyrimidin-2-yl]amino}phenyl)sulfoximide
(RS)—S-ethyl-S-(4-{[4-{[(R)-2-hydroxy-1,2-dimethylpropyl]amino}-5-(trifluoromethyl)pyrimidin-2-yl]amino}phenyl)sulfoximide
(RS)—S-(4-{[4-{[(1R,2R)-2-hydroxy-1-methylpropyl]amino}-5-(trifluoromethyl)pyrimidin-2-yl]amino}phenyl)-S-methylsulfoximide
(RS)—S-(4-{[4-{[(1R)-2-hydroxy-1,2-dimethylpropyl]amino}-5-(trifluoromethyl)pyrimidin-2-yl]amino}phenyl)-S-methylsulfoximide.

4. (canceled)

5. The method according to claim 1, wherein the tumor disorder is selected from
breast carcinomas, pancreas carcinomas, kidney carcinomas, malignant melanomas and other skin tumors, small-cell bronchial carcinomas, non-small-cell bronchial carcinomas, colorectal carcinomas, ovarial carcinomas, cervix carcinomas, prostate carcinomas, leukemias and lymphomas.

6. The method according to claim 1, wherein the tumor disorder is selected from
breast carcinomas, ovarial carcinomas, colorectal carcinomas, small-cell bronchial carcinomas and cervix carcinomas.

7. The method according to claim 1, wherein the compound is (RS)—S-(4-{[4-{[(1R,2R)-2-hydroxy-1-methylpropyl]oxy}-5-(trifluoromethyl)pyrimidin-2-yl]amino}phenyl)-S-methylsulfoximide
or a physiologically acceptable salt, diastereomer or enantiomer thereof,
and the tumor disorder is selected from multidrug-resistant cervix carcinomas, colorectal carcinomas, small-cell bronchial carcinomas, breast carcinomas and cisplatin-resistant ovarial carcinomas.

8. (canceled)

9. The method according to claim 1, wherein the compound is (RS)—S-(4-{[4-{[(1R,2R)-2-Hydroxy-1-methylpropyl]oxy}-5-(trifluoromethyl)pyrimidin-2-yl]amino}phenyl)-S-methylsulfoximide or a physiologically acceptable salt, diastereomer or enantiomer thereof.
